(19) United States
(12) Patent Application Publication
(10) Pub. No.: US 2010/0010533 A1
(43) Pub. Date: Jan. 14, 2010

(54) VARIABLE STRENGTH EMBOLIZATION COIL

(75) Inventors: JESSICA L. BURKE, Bloomington, IN (US); Jeffry S. Melsheimer, Springville, IN (US)

Correspondence Address: BRINKS HOFER GILSON & LIONE/CHICAGO/COOK, PO BOX 10395, CHICAGO, IL, 60610, US

(73) Assignee: COOK INCORPORATED, BLOOMINGTON, IN (US)

(21) Appl. No.: 12/171,900

(22) Filed: Jul. 11, 2008

(51) Int. Cl. A61M 29/00

(52) U.S. Cl. 606/200

(57) ABSTRACT

An occluding device having variable stiffness for occluding fluid flow through a lumen of a body vessel. The device comprises a wire having a first end and a second end. The wire is formed with a tapered diameter defined by a continuously decreasing diameter from the first end to the second end. The tapered wire is wound into a primary coil having a primary shape. The primary coil may then be wound into a secondary shape or received within a secondary coil which conforms to the primary shape of the primary coil. The tapered diameter provides the device with a continuously decreasing stiffness from a first end to a second end.

### BACKGROUND OF THE INVENTION

1. Field of Invention

The present invention relates to medical devices. More particularly, the invention relates to occluding devices and methods of occluding fluid flow through a body vessel.

2. Background

Embolization coils have been used as a primary occluding device for treatment of various arteriovenous malformations (AVM) and varicoceles, as well as for many other arteriovenous abnormalities in the body. Occluding devices are also used to repair abnormal shunts between arteries and veins, prevent or reduce blood flow to tumors, stop hemorrhaging as a result of trauma, and stabilize aneurysms to prevent rupture. Embolization coils, for example pushable fibered coils, may be configured in a variety of sizes with varying diameters and may be made of several different materials including stainless steel and platinum. Occlusion devices may vary for differing purposes, e.g., to hold the device in place within a cavity or vessel and to pack the device within the vessel for enhanced occlusion.

Although current coils are adequate, such coils may be improved for more effective occlusion of fluid flow through a lumen of a body vessel. Many medical procedures for occluding blood flow through an artery or vein require a number of coils, since a single coil or two may not be sufficient to effectively occlude blood flow through a lumen of an artery or vein. For example, a coil having greater stiffness or rigidity may be introduced into a blood vessel and various coils of decreasing stiffness or rigidity may follow behind the stiffer coil. This procedure may involve an undesirable amount of additional time and increased costs associated with manufacturing and deploying a number of different coils.

### BRIEF SUMMARY OF THE INVENTION

The present invention provides an improved occluding device and an improved method of occluding fluid flow through a lumen of a body vessel. The occluding device comprises a coil formed from a wire having a variable stiffness.

In one embodiment, the occluding device includes an elongate wire having a proximal end, a distal end, and a central axis extending between the proximal and distal ends. The wire is formed with a tapered diameter, the proximal end having a smaller diameter than the distal end. The tapered diameter is defined by a gradually or continuously decreasing diameter along its central axis from the distal end to the proximal end. The tapered wire is coiled into a primary shape defined by a linear longitudinally extending coil. The coiled wire in the primary shape is helically wound into a secondary shape defined by a spiral shaped coil having a plurality of axially spaced loops. The tapered diameter of the coiled wire provides the device with a continuously decreasing stiffness from the distal end to the proximal end.

In another embodiment, the occluding device includes an elongate first wire having a proximal end, a distal end, and a central axis extending between the proximal and distal ends. The first wire is formed with a tapered diameter, the proximal end having a smaller diameter than the distal end. The tapered diameter is defined by a gradually or continuously decreasing diameter along its central axis from the distal end to the proximal end. The tapered wire is coiled into a primary shape defined by a spiral shaped first coil having a plurality of axially spaced loops. An elongate second wire including a proximal end and a distal end is wound into a second coil having a primary shape defined by a linear longitudinally extending coil. The second coil in its primary shape receives the first wire and conforms to the primary shape of the coiled first wire thereby forming a secondary shape of the second coil, which is defined by a spiral shaped second coil having a plurality of axially spaced loops. In this embodiment, the first wire serves as an inner mandrel within the second coil. The tapered diameter of the coiled first wire provides the device with a continuously decreasing stiffness from the distal end to the proximal end.

In yet another embodiment, the occluding device includes an elongate wire having a first end, a second end, and a central axis extending between the first and second ends. The wire tapers along its central axis from a larger diameter at the first end to a smaller diameter at the second end. The tapered wire is coiled into a primary shape defined by a linear longitudinally extending coil. The coiled wire in the primary shape is helically wound into a secondary shape defined by a spiral shaped coil having a plurality of axially spaced loops. The tapered wire provides the device with a continuously decreasing stiffness from the first end to the second end.

In still another embodiment, the occluding device includes an elongate first wire having a first end, a second end, and a central axis extending between the first and second ends. The first wire tapers along its central axis from a larger diameter at the first end to a smaller diameter at the second end. The tapered first wire is coiled into a primary shape defined by a spiral shaped first coil having a plurality of axially spaced loops. An elongate second wire including a proximal end and a distal end is wound into a second coil having a primary shape defined by a linear longitudinally extending coil. The second coil in its primary shape receives the first wire and conforms to the primary shape of the coiled first wire thereby forming a secondary shape of the second coil, which is defined by a spiral shaped second coil having a plurality of axially spaced loops. In this embodiment, the first wire serves as an inner mandrel within the second coil. The tapered first wire provides the device with a continuously decreasing stiffness from the distal end to the proximal end.

The present invention further includes an improved embolization kit for occluding fluid flow through a body vessel. The kit comprises an occluding device in accordance with one embodiment of the present invention as well as a guide catheter. An inner catheter having proximal and distal ends is configured to be passed through the guide catheter to position the inner catheter in the body vessel and to deploy the occluding device. The inner catheter has a hub adjacent the proximal end.

The present invention also includes an improved method for occluding fluid flow through a body vessel. The method comprises forming a variable stiffness occluding device and deploying the occluding device into a lumen of the body vessel. Forming the variable stiffness occluding device includes tapering an elongate first wire having a first end, a second end, and a central axis extending between the first and second ends. The first wire is tapered to form a continuously decreasing diameter along its central axis from the first end to the second end. The tapered first wire is then coiled. The tapered first wire provides the device with a continuously decreasing stiffness from the first end to the second end.

In one method in accordance with the present invention, coiling the tapered first wire includes winding the tapered first wire into a primary shape defined by a linear longitudinally extending coil and winding the coiled first wire in its primary shape into a secondary shape defined by a spiral shaped coil having a plurality of axially spaced loops.

In another method in accordance with the present invention, coiling the tapered first wire includes winding the tapered first wire into a primary shape defined by a spiral shaped coil having a plurality of axially spaced loops. In this embodiment, forming the variable stiffness occluding device further includes coiling an elongate second wire having a first end and a second end into a second coil having a primary shape defined by a linear longitudinally extending coil. The second coil in its primary shape includes a second central axis extending between first and second ends of the second coil. The longitudinally extending second coil receives the first wire and conforms to the primary shape of the coiled first wire thereby defining a secondary shape of the second coil. The first and second axes coincide and the first and second ends of the first wire are adjacent the first and second ends of the second coil, respectively, when the second coil receives the first wire and forms its secondary shape defined by a spiral shaped second coil having a plurality of axially spaced loops.

Further objects, features, and advantages of the present invention will become apparent from consideration of the following description and the appended claims when taken in connection with the accompanying drawings.

### BRIEF DESCRIPTION OF THE DRAWINGS

FIG. 1 is a partial side view of a pre-coiled tapered wire in accordance with an embodiment of the present invention;

FIG. 2 is a partial side perspective view of the tapered wire of FIG. 1 coiled into a primary shape in accordance with one embodiment of the present invention;

FIG. 3a is a partial side view of a coiled second wire in accordance with one embodiment of the present invention;

FIG. 3b is a partial side perspective view of an occluding device in accordance with the embodiments of FIGS. 2 and 3a;

FIG. 4a is a partial side view of a coiled second wire in accordance with another embodiment of the present invention;

FIG. 4b is a partial side perspective view of an occluding device in accordance with the embodiments of FIGS. 2 and 4a;

FIG. 5a is a partial side view of a tapered wire coiled into a primary shape in accordance with another embodiment of the present invention;

FIG. 5b is a partial side perspective view of an occluding device in accordance with the embodiment of FIG. 5a;

FIG. 6 is a cross-sectional environmental view of an occluding device deployed in a body vessel;

FIG. 7a is an exploded view of an embolization kit in accordance with an embodiment of an occluding device of the present invention;

FIG. 7b is a side view of an embolization kit in accordance with an embodiment of the present invention; and

FIG. 8 is a flowchart for a method of occluding fluid flow through a body vessel in accordance with one example of the present invention.

### DETAILED DESCRIPTION OF THE INVENTION

The following provides a detailed description of currently preferred embodiments of the present invention. The description is not intended to limit the invention in any manner, but rather serves to enable those skilled in the art to make and use the invention.

The present invention generally provides an occluding device used for transcatheter embolization and having variable stiffness or rigidity to eliminate the need for an additional coil of yet another strength, and to provide an improved occlusion of fluid flow through the vessel. The occluding device is an embolization coil preferably used to occlude fluid flow through a lumen of a body vessel such as for an occlusion of an arteriovenous malformation (AVM). The occluding device comprises a primary coil having a continuously changing stiffness along the length of the coil from the distal end to the proximal end. Preferably, the primary coil is formed into a helical shape and further defines a secondary coil. To further facilitate occlusion of fluid flow the occluding device may comprise fibers attached between loops of the primary coil and extending therefrom.

The occluding device also may be used for treatment of renal arteriovenous malfunction (AVM), pulmonary AVM, vascular tumors, low-flow fistulas, trauma related hemorrhages, and visceral vasculature defects including varicoceles, aneurysms, and selected telangiectasias. For example, treatment of visceral vasculature defects may include but are not limited to embolotherapy on gastroduogenal hemorrhages, hepatic aneurysms, celiac aneurysms, internal iliac aneurysms, and internal spermatic varicoceles.

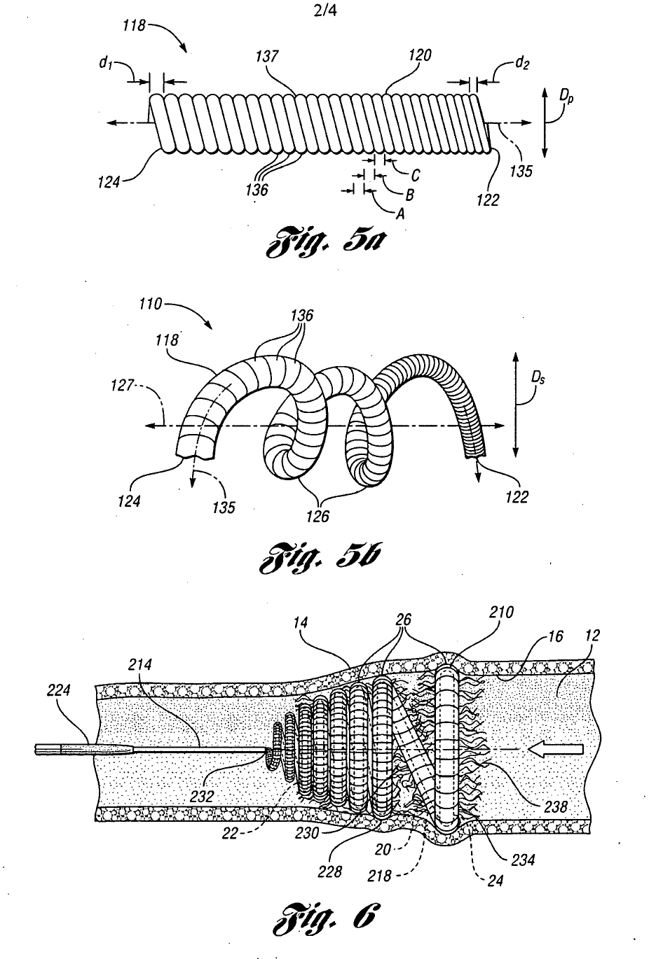

Referring to FIGS. 3b, 4b, 5b, and 6, at least one embodiment of an occluding device in accordance with the present invention is provided. FIG. 6 illustrates an occluding device 210 in a deployed state for occlusion of fluid flow through a lumen 12 of a body vessel 14. As shown, the occluding device 210 is positioned to engage an inner wall 16 of the body vessel 14 and comprises a primary coil 218 and a secondary coil 228.

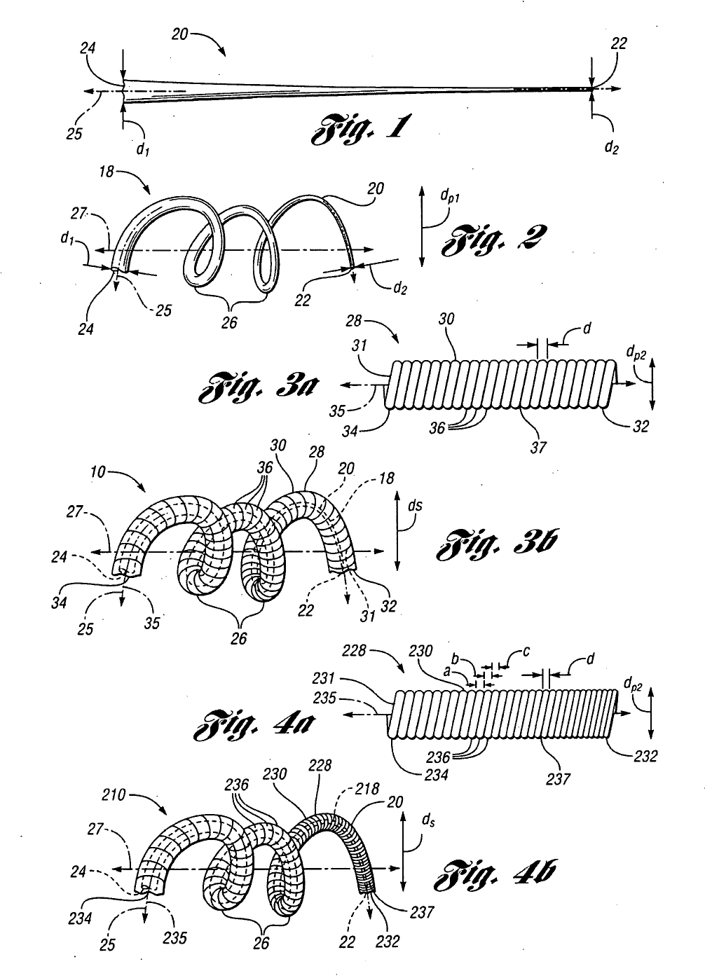

In one embodiment, a wire 20 is tapered and wound into a primary coil 18 of an occluding device 10. As illustrated in FIG. 1, the wire 20 includes a proximal end 22, a distal end 24, and a central axis 25 extending between the proximal and distal ends 22, 24. The wire 20 is tapered along the central axis 25 from the distal end 24 to the proximal end 22 defining a first diameter d₁ at the distal end 24, a second diameter d₂ at the proximal end 22, and a gradually or continuously changing diameter from the distal end 24 to the proximal end 22. For example, every successive point along the wire 20 proximal the distal end 24 has a diameter successively smaller than d₁ and every successive point along the wire 20 distal the proximal end 22 has a diameter successively larger than d₂.

As shown in FIG. 1, the wire 20 is tapered along its entire length, from the distal end 24 having the largest diameter (i.e., the greatest stiffness) to the proximal end 22 having the smallest diameter (i.e., the lowest stiffness), forming a continuously changing diameter along the length of the wire 20. The wire 20 may be tapered via centerless grinding, electrolytic tapering, or any other technique suitable for providing a smooth, controlled decrease in diameter along the length of the wire 20 between opposing ends 22, 24.

In this embodiment, the tapered wire 20 is wound into the primary coil 18 having variable stiffness along the length of the coil 18. Preferably, the tapered wire 20 is curled or coiled about a longitudinal axis 27 into a primary coil 18 having a primary shape defined by a plurality of turns or loops 26 wound about the longitudinal axis 27 of the primary coil 18 and axially spaced apart by a predetermined distance. The plurality of loops 26 defines a cross-sectional area formed axially along the primary coil 18. In this embodiment, the predetermined distance may be in the range of around 0 to 5 millimeters curl space. Curl space is defined as the distance between two loops 26 of the primary coil 18. As shown in FIG. 2, the primary shape of the coiled tapered wire 20 or primary coil 18 is spiral shaped. The diameter of the primary coil 18 in the primary shape (i.e., the primary diameter dₚ₁) may be generally constant, resulting in a generally linear coil. Alternatively, the primary diameter dₚ₁ of the primary coil 18 may be varied along the length of the primary coil 18. For example, the primary shape of the primary coil 18 may include a changing primary diameter dₚ₁ defined by a plurality of radially expanding loops 26 forming a conically helically shaped coil, an example of which is shown in FIG. 6.

The tapered wire 20 may be coiled into the primary coil 18 by any apparatus known in the art, such as a roller deflecting apparatus, a mandrel apparatus, or any other suitable means. For example, the tapered wire 20 may be wound about a mandrel and heat set to form its spiral shape. Alternatively, the tapered wire 20 may be wound about a longitudinally tapered mandrel and heat set to form a conically helically shaped coil.

As illustrated in FIG. 2, tapering the wire 20 along its length before coiling the wire 20 provides the primary coil 18 with a tapered diameter from the distal end 24 having a larger outer diameter d₁ to the proximal end 22 having a smaller outer diameter d₂, and a gradually or continuously decreasing outer diameter from the distal end 24 to the proximal end 22 such that every successive point along the primary coil 18 proximal the distal end 24 has a diameter successively smaller than d₁ and every successive point along the primary coil 18 distal the proximal end 22 has a diameter successively larger than d₂.

In this embodiment, the tapered wire 20 is defined by a successive or a continuous decline in the diameter of the wire 20. Forming the primary coil 18 from this single tapered wire 20 provides a continuous decline in the diameter of the primary coil 18 along the entire length of the primary coil 18 as opposed to stepped or segmented regions of decreasing wire/coil diameter. Thus, the initial tapering of the wire 20 substantially eliminates the risk of potential failure or kink points which result from forming a variable stiffness coil by, for example, soldering multiple wires of differing diameters together. Coils having these hinge or kink points have an undesirable innate tendency to bend, whereas the primary coil 18 formed from the tapered wire 20 has a smoothly transitioned decrease in diameter/stiffness and does not have an innate tendency to bend. The continuously decreasing diameter of the coiled tapered wire 20 (i.e., the primary coil 18) provides the device 10 with a continuously decreasing stiffness.

As illustrated in FIG. 3a, a wire 30 having a proximal end 32 and a distal end 34 is wound about a longitudinal axis 35 into a secondary coil 28 having proximal and distal ends 32, 34. The longitudinal axis 35 forms the central axis of the secondary coil 28. In this embodiment, the wire 30 has a generally constant diameter and thus the secondary coil 28 has a generally constant diameter d, shown in FIG. 3a.

In the embodiment shown in FIG. 4a, a wire 230 has a proximal end 232, a distal end 234, and a tapered diameter from the distal end 234 to the proximal end 232 similar to the tapered diameter of the wire 20. The wire 230 is wound about a longitudinal axis 235 into a secondary coil 228 having proximal and distal ends 232, 234. The longitudinal axis 235 forms the central axis of the secondary coil 228. The wire 230 has a tapered diameter and thus, the secondary coil 228 has a tapered diameter d, as shown in FIG. 4a. In this embodiment, the secondary coil 228 includes a tapered diameter d from the distal end 234 having a larger diameter to the proximal end 232 having a smaller diameter, and a gradually or continuously decreasing diameter from the distal end 234 to the proximal end 232 such that every successive point along the secondary coil 228 proximal the distal end 234 has a diameter successively smaller than the diameter at the distal end 234 and every successive point along the secondary coil 228 distal the proximal end 232 has a diameter successively larger than the diameter at the proximal end (i.e., a>b>c).

Preferably, the wire 30, 230 is wound about the longitudinal axis 35, 235 into a longitudinally extending secondary coil 28, 228 having an inner lumen 31, 231 that is configured to receive the tapered wire 20 disposed therethrough. In this embodiment, the secondary coil 28, 228 has a generally linear primary shape and includes a plurality of tightly spaced turns 36, 236 with minimal, if any spacing 37, 237 therebetween. The generally linear primary shape is defined by a generally constant primary diameter dₚ₂. The wire 30, 230 is wound into the secondary coil 28, 228 by any apparatus known in the art, such as a roller deflecting apparatus, a mandrel apparatus, or any other suitable means. For example, the wire 30, 230 may be wrapped around a mandrel and heat set to form its primary shape.

As illustrated in FIGS. 3b and 4b, the tapered wire 20 is received within the lumen 31, 231 of the secondary coil 28, 228, wherein the coiled tapered wire 20 (i.e., the primary coil 18) provides the secondary coil 28, 228 with its secondary shape. As illustrated in FIG. 2, the tapered wire 20 is initially curled or coiled into the primary coil 18. In one embodiment, the linear longitudinally extending secondary coil 28, 228 is threaded or slid over the tapered wire 20 in its coiled configuration (i.e., the primary coil 18 shown in FIG. 2).

In this embodiment, the central axis 35, 235 of the secondary coil 28, 228 is aligned with the central axis 25 of the coiled tapered wire 20. With the distal end 34, 234 of the secondary coil 28, 228 adjacent the proximal end 22 of the coiled tapered wire 20, the secondary coil 28, 228 slides over the coiled tapered wire 20 until the distal end 34, 234 of the secondary coil 28, 228 meets the distal end 24, 224 of the coiled tapered wire 20, as shown in FIGS. 3b and 4b. In this embodiment, the secondary coil 28, 228 conforms to the shape of the coiled tapered wire 20 as the overlying secondary coil 28, 228 moves along the plurality of loops 26 of the coiled tapered wire 20, coiling about the longitudinal axis 27, and thus forming the secondary shape of the secondary coil 28, 228.

In another embodiment, the coiled tapered wire 20 may be straightened before being received within the lumen 31 of the linear longitudinally extending secondary coil 28, 228. In this embodiment, the central axis 35, 235 of the secondary coil 28, 228 is aligned with the central axis 25 of the tapered wire 20. With the distal end 34, 234 of the secondary coil 28, 228 adjacent the proximal end 22 of the tapered wire 20, the secondary coil 28, 228 slides over the straightened tapered wire 20 until the distal end 34, 234 of the secondary coil 28, 228 meets the distal end 24 of the tapered wire 20. Thereafter, the tapered wire 20 within the secondary coil 28, 228 returns to its coiled configuration (i.e., the primary coil 18) causing the secondary coil 28, 228 to take the shape of the primary coil 18, both the primary coil 18 and the secondary coil 28, 228 coiling about the longitudinal axis 27, thus forming the secondary shape of the secondary coil 28, 228.

Thus, the coiled tapered wire 20 (i.e., primary coil 18) provides the secondary coil 28, 228 with its secondary shape defined by the plurality of axially spaced loops 26. The tapered diameter of the wire 20 provides the secondary coil 28, 228 with its variable strength (i.e., continuously decreasing stiffness from the distal 34, 234 end to the proximal end 32, 232). Thus, the coiled tapered wire 20 serves as an inner mandrel within the secondary coil 28, 228 and provides the secondary coil 28, 228 with a gradually decreasing stiffness from the distal end 34, 234 to the proximal end 32, 232 resulting in a variable strength occluding device 10, 210.

In this embodiment, the larger diameter at the distal end 24 of the coiled tapered wire 20 disposed within the secondary coil 28, 228 establishes a greater stiffness or rigidity at the distal end 34, 234 of the secondary coil 28, 228, which facilitates anchoring or engagement of the occluding device 10, 210 within the body vessel 14 and prevents the occluding device 10, 210 from migration by retaining its position along the inner wall 16 of the body vessel 14. The more flexible proximal end 22 of the coiled tapered wire 20, and thus the proximal end 32, 232 of the secondary coil 28, 228, serves to pack behind the more rigid distal end 34, 234 inside the lumen 12 of the body vessel 14.

In a preferred embodiment, the diameter of the wires 20, 30 is between around 0.0005 and 0.008 inch. Larger diameter wire (0.003 to 0.008 inch) may be desired for very specific indications where occlusion is needed at a high volume flow rate site. For example, the wire 20 may taper from a larger diameter at the distal end 24 of around 0.006 inch to a smaller diameter at the proximal end 22 of around 0.002 inch. The primary 18 and secondary coil 28, 228 may have a length of between about 3 to 20 centimeters. The secondary coil 28, 228 in its primary shape may have a primary diameter dₚ₂ of between about 0.010 and 0.035 inch. In this embodiment, since the secondary coil 28, 228 is configured to receive the tapered wire 20 (i.e., the primary coil 18), the primary diameter dₚ₂ of the secondary coil 28, 228 is dimensioned to receive the larger diameter d₁ of the tapered wire 20.

In a preferred embodiment, the outer diameter of the secondary coil 28, 228 in its secondary shape (i.e., the secondary diameter dₛ) may range between about 3 to 15 millimeters. Preferably, the secondary diameter dₛ at the distal end 34, 234 is selected so that, when unconstrained, it is slightly larger than the body vessel 14 into which it is placed, allowing the device 10, 210 to engage the inner wall 16 of the lumen 12. The secondary shape of the secondary coil 28, 228 is shaped by the primary shape of the primary coil 18, and thus the secondary diameter dₛ corresponds with the primary diameter dₚ₁ of the primary coil 18 and may be generally constant or varied. Alternatively, the secondary shape may be non-linear and include a plurality of radially expanding loops 26 (i.e., a radially increasing secondary diameter dₛ) forming a conically helically shaped coil, an example of which is shown in FIG. 6. All of the dimensions here are provided only as guidelines and are not critical to the invention.

In yet another embodiment, as shown in FIG. 6, to assist in occluding fluid flow through the lumen 12 of the body vessel 14, the occluding device 210 may further includes a series of fibers 238 attached between loops 26 of the secondary coil 228 and extending therefrom. The fibers 238 may be attached to the wire 230 before or after the wire 230 is coiled into the secondary coil 228. In a preferred embodiment, the fibers 238 include strands comprising a synthetic polymer such as polyester textile fiber, e.g., DACRON™. As desired, the strands may be positioned between adjacent loops, alternating loops, alternating double loops, or any desired configuration.

In a preferred embodiment, the proximal 32, 232 and/or the distal end 34, 234 of the secondary coil 28, 228 includes a cap or is soldered or welded to present a rounded or smooth surface, which will not catch on the interior surface of the guiding catheter or provide a source of trauma for the vasculature.

The tapered wire 20 may be attached to the secondary coil 28, 228 via adhesive bonding, soldering, welding, friction connection, compression fit, and crimping. However, any other suitable processes known in the art for attaching coils may also be used to attach the tapered wire 20 within the secondary coil 28, 228.

Preferably, the wires 20, 30 making up the primary 18 and secondary coils 28, 228 are made of any suitable material that will result in a device 10 capable of being percutaneously inserted and deployed within a body cavity. Examples of preferred materials include metallic materials, such as stainless steel, platinum, iron, iridium, palladium, tungsten, gold, rhodium, rhenium, and the like, as well as alloys of these metals. Other suitable materials include superelastic materials, a cobalt-chromium-nickel-molybdenum-iron alloy, a cobalt chrome-alloy, stress relieved metal, nickel-based superalloys, such as Inconel, or any magnetic resonance imaging (MRI) compatible material, including materials such as a polypropylene, nitinol, titanium, copper, or other metals that do not disturb MRI images adversely. The wires 20, 30 may also be made of radiopaque material, including tantalum, barium sulfate, tungsten carbide, bismuth oxide, barium sulfate, and cobalt alloys.

Further, the wires 20, 30 making up the primary 18 and secondary coils 28, 228 may be fabricated from shape memory materials or alloys, such as superelastic nickel-titanium alloys. An example of a suitable superelastic nickel-titanium alloy is Nitinol, which can “remember” and recover a previous shape. Nitinol undergoes a reversible phase transformation between a martensitic phase and an austenitic phase that allows it to “remember” and return to a previous shape or configuration. For example, compressive strain imparted to the coils 18, 28, 228 in the martensitic phase to achieve a low-profile delivery configuration may be substantially recovered during a reverse phase transformation to austenite, such that the coils 18, 28, 228 expand to a “remembered” (e.g., deployed) configuration at a treatment site in a vessel. Typically, recoverable strains of about 8-10% may be obtained from superelastic nickel-titanium alloys. The forward and reverse phase transformations may be driven by a change in stress (superelastic effect) and/or temperature (shape memory effect).

Slightly nickel-rich Nitinol alloys including, for example, about 51 at. % Ni and about 49 at. % Ti are known to be useful for medical devices which are superelastic at body temperature. In particular, alloys including 50.6-50.8 at. % Ni and 49.2-49.4 at. % Ti are considered to be medical grade Nitinol alloys and are suitable for the present coils 18, 28, 228. The nickel-titanium alloy may include one or more additional alloying elements.

In a preferred embodiment, the tapered wire 20 (i.e., primary coil 18) is made of nitinol or stainless steel and the wire 30 (i.e., secondary coil 28, 228) is made of palladium. A primary coil 18 made of nitinol, for example, may provide many clinical advantages. After the nitinol tapered wire 20 is initially curled or coiled into the primary coil 18, it is effectively straightened-out in order to thread or slide the secondary coil 28, 228 over it. Nitinol's super-elastic properties allow the tapered wire 20 to recover from the straightening strain and later return to its coiled primary shape.

Alternatively, the nitinol tapered wire 20 may be curled or coiled into the primary coil 18 and heat-set such that after it is effectively straightened for sliding the secondary coil 28, 228 over it, the device 10, 210 (i.e., the tapered wire 20 within the secondary coil 28, 228) may be heated to a predetermined activating temperature to induce the shape-memory property of the nitinol tapered wire 20 and cause it to return to the coiled configuration (i.e., primary shape) of the primary coil 18, thus causing the secondary coil 28, 228 to take on the primary shape of the primary coil 18.

In this embodiment, the device 10, 210 may be stored in the straightened configuration for delivery to the interventionalist. As the device 10, 210 is introduced into the body, body heat activates the shape-memory property of the nitinol tapered wire 20 within the secondary coil 28, 228 and causes the tapered wire 20 to return to the primary shape of the primary coil 18, and thus causes the secondary coil 28, 228 to take on the primary shape of the primary coil 18. The nitinol tapered wire 20 thus provides the secondary coil 28, 228 with its secondary shape and variable stiffness due to the tapered diameter of the wire 20, therefore serving as an inner mandrel within the secondary coil 28, 228.

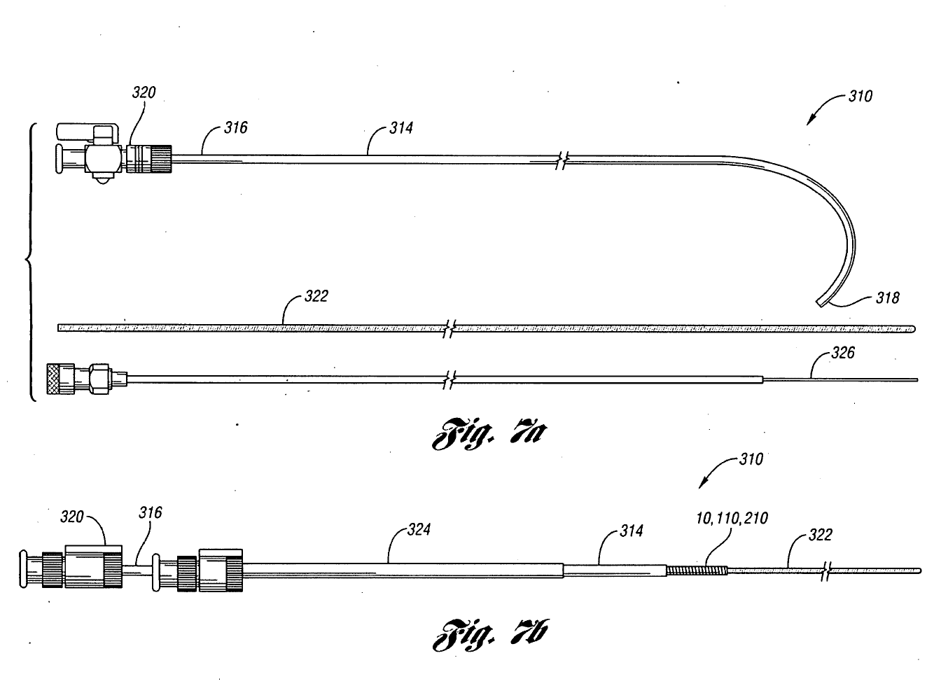

Referring to FIGS. 5a and 5b, another example of an occluding device 110 in accordance with the present invention is provided. As shown, the occluding device 110 comprises a coil 118 wound from a wire 120, similar to the wire 20 in FIG. 1, having a proximal end 122, a distal end 124, and a central axis extending between the proximal and distal ends 122, 124. In this embodiment, the wire 120 is tapered along its central axis from the distal end 124 to the proximal end 122 defining a larger diameter d₁ at the distal end 124, a smaller diameter d₂ at the proximal end 122, and a gradually or continuously changing diameter from the distal end 124 to the proximal end 122. For example, every successive point along the wire 120 proximal the distal end 124 has a diameter successively smaller than d₁ and every successive point along the wire 120 distal the proximal end 122 has a diameter successively larger than d₂.

In this embodiment, the wire 120 is tapered along its entire length, from the distal end 124 having the largest diameter (i.e., the greatest stiffness) to the proximal end 122 having the smallest diameter (i.e., the lowest stiffness), forming a continuously changing diameter along the length of the wire 120. The wire 120 may be tapered via centerless grinding, electrolytic tapering, or any other technique suitable for providing a smooth, controlled decrease in diameter along the length of the wire 120 between opposing ends 122, 124.

In this embodiment, the tapered wire 120 is wound about a longitudinal axis 135 into a longitudinally extending coil 118 having a variable stiffness along the length of the coil 118, as illustrated in FIG. 5a. The longitudinal axis 135 forms the central axis of the coil 118. Preferably, the wire 120 is wound into a coil 118 having a generally linear primary shape including a plurality of tightly spaced turns 136 with minimal, if any, spacing 137 therebetween. The generally linear primary shape is defined by a generally constant primary diameter Dₚ. The wire 120 may be wound into the coil 118 by any apparatus known in the art, such as a roller deflecting apparatus, a mandrel apparatus, or any other suitable means. For example, the wire 120 may be wrapped around a mandrel and heat set to form its primary shape.

In this embodiment, tapering the wire 120 before coiling the wire 120 into the coil 118 provides the coil 118 with a tapered diameter from the distal end 124 having a larger diameter d₁ to the proximal end 122 having a smaller diameter d₂, and a gradually or continuously decreasing diameter from the distal end 124 to the proximal end 122 such that every successive point along the coil 118 proximal the distal end 124 has a diameter successively smaller than d₁ and every successive point along the coil 118 distal the proximal end 122 has a diameter successively larger than d₂ (i.e., A>B>C).

In this embodiment, the tapered wire 120 is defined by a successive or continuous decline in the diameter of the wire 120. Forming the coil 118 from this single tapered wire 120 provides a continuous decline in the diameter of the primary coil 18 along the entire length of the coil 118 as opposed to stepped or segmented regions of decreasing wire/coil diameter. Thus, the initial tapering of the wire 120 substantially eliminates the risk of potential failure or kink points which result from forming a variable stiffness coil from multiple wires of differing diameters which are, for example, soldered together. Coils having these hinge or kink-points have an undesirable tendency to bend in a very localized region, whereas the coil 118 formed from the tapered wire 120 has a smoothly transitioned decrease in diameter/stiffness and does not have an innate tendency to bend sharply. The continuously decreasing diameter of the coiled tapered wire 120 (i.e., the coil 118) provides the device 110 with a continuously decreasing stiffness.

As shown in FIG. 5b, the coil 118 in the primary shape is helically wound about a longitudinal axis 127 into a secondary shape defined by a plurality of turns or loops 126 which define a cross-sectional area formed axially along the coil 118 in the secondary shape. The loops 126 may be axially spaced apart by a predetermined distance. In this embodiment, the predetermined distance may be around 0 to about 5 millimeters curl space. Curl space is defined as the distance between two loops 126 of the coil 118 in the secondary shape. As shown in FIG. 5b, the coil 118 in the secondary shape is spiral shaped. The diameter of the coil 118 in the secondary shape (i.e., the secondary diameter Dₛ) may be generally constant, resulting in a generally linear coil. Alternatively, the secondary diameter Dₛ may be varied along the length of the coil 118. For example, the secondary shape of the coil 118 may include a plurality of radially expanding loops 26 (i.e., a radially increasing secondary diameter Dₛ) forming a conically helically shaped coil 118.

In this embodiment, the coil 118 in the primary shape may be wound into the secondary shape by any apparatus known in the art, such as a roller deflecting apparatus, a mandrel apparatus, or any other suitable means. For example, the coil 118 may be wound about a mandrel and heat set to form its secondary shape. Alternatively, the coil 118 may be wound about a longitudinally tapered mandrel and heat set to form a conically helically shaped coil, similar to the occluding device 210 illustrated in FIG. 6.

The tapered diameter of the coiled tapered wire 120 (i.e., the coil 118) provides the device 110 with its variable strength (i.e., continuously decreasing stiffness from the distal end 124 to the proximal end 122). The larger diameter of the distal end 124 of the coil 118 establishes a greater stiffness or rigidity, which facilitates anchoring or engagement of the occluding device 110 within the body vessel 14 and prevents the occluding device 110 from migration by retaining its position along the inner wall 16 of the body vessel 14. The more flexible proximal end 122 of the coil 118 serves to pack behind the more rigid distal end 124 inside the lumen 12 of the body vessel 14.

In a preferred embodiment, the diameter of the wire 120 is preferably between around 0.0005 and 0.008 inch. Larger diameter wire (0.003 to 0.008 inch) may be desired for very specific indications where occlusion is needed at a high volume flow rate site. For example, the wire 120 may taper from a larger diameter at the distal end 124 of around 0.006 inch to a smaller diameter at the proximal end 122 of around 0.002 inch. The coil 118 may have a length of between about 3 to 20 centimeters. The coil 118 in its generally linear primary shape may have a primary diameter Dₚ of between about 0.010 and 0.035 inch. In a preferred embodiment, the secondary diameter Dₛ of the coil 118 may range between about 3 to 15 millimeters. Preferably, the secondary diameter Dₛ at the distal end 134 is selected so that, when unconstrained, it is slightly larger than the body vessel 14 into which it is placed, allowing the device 110 to engage the inner wall 16 of the lumen 12. All of the dimensions here are provided only as guidelines and are not critical to the invention.

Additionally, to assist in occluding fluid flow through the lumen 12 of the body vessel 14, the occluding device 110 may includes a series of fibers attached between loops 126 of the coil 118 and extending therefrom. The fibers may be attached to the wire 120 before or after the wire 120 is coiled into the coil 118. In one embodiment, the fibers include strands comprising a synthetic polymer such as polyester textile fiber, e.g., DACRON™. As desired, the strands may be positioned between adjacent loops, alternating loops, alternating double loops, or any desired configuration. In a preferred embodiment, the proximal 122 and/or the distal end 124 of the coil 118 includes a cap or is soldered or welded to present a rounded or smooth surface, which will not catch on the interior surface of the guiding catheter or provide a source of trauma to the vasculature.

Preferably, the wire 120 making up the coil 118 is made of any suitable material that will result in a device 110 capable of being percutaneously inserted and deployed within a body cavity. Examples of preferred materials include metallic materials, such as stainless steel, platinum, iron, iridium, palladium, tungsten, gold, rhodium, rhenium, and the like, as well as alloys of these metals. Other suitable materials include superelastic materials, a cobalt-chromium-nickel-molybdenum-iron alloy, a cobalt chrome-alloy, stress relieved metal, nickel-based superalloys, such as Inconel, or any magnetic resonance imaging (MRI) compatible material, including materials such as a polypropylene, nitinol, titanium, copper, or other metals that do not disturb MRI images adversely. The wire 120 may also be made of radiopaque material, including tantalum, barium sulfate, tungsten carbide, bismuth oxide, barium sulfate, and cobalt alloys.

Further, the wire 120 may be fabricated from shape memory materials or alloys, such as superelastic nickel-titanium alloys. An example of a suitable superelastic nickel-titanium alloy is Nitinol, which can “remember” and recover a previous shape. Nitinol undergoes a reversible phase transformation between a martensitic phase and an austenitic phase that allows it to “remember” and return to a previous shape or configuration. For example, compressive strain imparted to the coils 118 in the martensitic phase to achieve a low-profile delivery configuration may be substantially recovered during a reverse phase transformation to austenite, such that the coil 118 expands to a “remembered” (e.g., deployed) configuration at a treatment site in a vessel. Typically, recoverable strains of about 8-10% may be obtained from superelastic nickel-titanium alloys. The forward and reverse phase transformations may be driven by a change in stress (superelastic effect) and/or temperature (shape memory effect).

Slightly nickel-rich Nitinol alloys including, for example, about 51 at. % Ni and about 49 at. % Ti are known to be useful for medical devices which are superelastic at body temperature. In particular, alloys including 50.6-50.8 at. % Ni and 49.2-49.4 at. % Ti are considered to be medical grade Nitinol alloys and are suitable for the present coil 118. The nickel-titanium alloy may include one or more additional alloying elements.

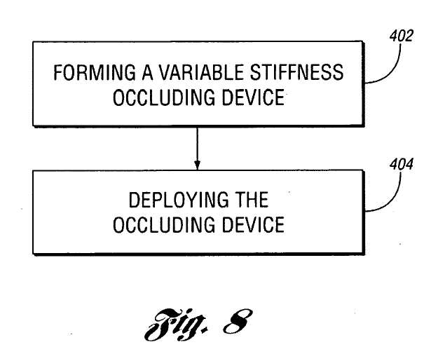

FIGS. 7a and 7b illustrate an embolization kit 310 which implements the occluding device 10, 110 in accordance with one embodiment of the present invention. As shown, the kit 310 includes an inner catheter 314 preferably made from a soft, flexible material such as silicone or any other suitable material. Generally, the inner catheter 314 has a proximal end 316, a distal end 318, and a plastic adapter or hub 320 to receive apparatus to be advanced therethrough. In this embodiment, the inside diameter of the inner catheter may range between 0.014 and 0.027 inch. The kit 310 further includes a guide wire 322 which provides the guide catheter 324 (discussed in more detail below) a path during insertion of the guide catheter 324 within a body cavity. The size of the wire guide is based on the inside diameter of the guide catheter 324.

In this embodiment, the kit 310 further includes a polytetrafluoroethylene (PTFE) guide catheter or sheath 324 for percutaneously introducing the inner catheter 314 in a body vessel 14. Of course, any other suitable material may be used without falling beyond the scope or spirit of the present invention. The guide catheter 324 may have a size of about 4-French to 8-French and allows the inner catheter 314 to be inserted therethrough to a desired location in the body cavity. The guide catheter 324 receives the inner catheter 314 and provides stability of the inner catheter 314 at a desired location of the body cavity. For example, the guide catheter 324 may stay stationary within a common visceral artery, e.g., a common hepatic artery, and add stability to the inner catheter 314 as the inner catheter is advanced through the guide catheter to a point of occlusion in a connecting artery, e.g., the left or right hepatic artery.

When the distal end 318 of the inner catheter 314 is at the point of occlusion in the body cavity, the occluding device is loaded at the proximal end 316 of the inner catheter 314 and is advanced through the inner catheter for deployment through the distal end 318. In this embodiment, a pushwire 326 is used to mechanically advance or push the occluding device through the inner catheter 314. The size of the push wire used depends on the diameters of the inner catheter. As mentioned above, when the device 10, 110 is deployed in the body vessel 14, the distal end 24, 124 of the coil 18, 118 serves to hold the coil in place along the inner wall 16 of the body vessel 14. The proximal end 22, 122 of the occluding device and the fibers 38 serve to occlude fluid flow by filling the lumen 12 of the body vessel 14.

In an alternative embodiment, an elongated releasing member (not shown) made be used instead of a pushwire 64. The elongated releasing member is similar to the pushwire 326 in that it may be advanced through the inner catheter 314 to deploy the device 10, 110 through the distal end 318. However, the elongated releasing member further includes a distal end configured for selectively engaging and/or disengaging with the device 10, 110. Once the device 10, 110 is deployed through the inner catheter 314, the elongated releasing member may be twisted or un-screwed to disengage the device 10, 110 from the elongated releasing member, thus releasing the device 10, 110 within the body vessel 14. Other suitable releasing devices known to those skilled in the art may also be used to advance and selectively deploy the occluding device 10 from the inner catheter 314.

It is to be understood that the embolization kit 310 described above is merely one example of a kit that may be used to deploy the occluding device in a body vessel. Of course, other kits, assemblies, and systems may be used to deploy any embodiment of the occluding device without falling beyond the scope or spirit of the present invention.

Referring to FIG. 8, a method of occluding fluid flow through a lumen of a body vessel is provided. The method includes forming a variable stiffness occluding device (402) as discussed in the forgoing paragraphs. Forming the variable stiffness occluding device includes tapering a first elongate wire. The first wire includes a first end, a second end, and a first central axis extending between the first and second ends. Tapering the first wire may include grinding, electrolytically tapering or performing any other suitable tapering technique to form a gradually or continuously decreasing diameter along the first central axis from the first end to the second end. The tapered first wire provides the device with a continuously decreasing stiffness from the first end to the second end.

The step of forming the variable strength occluding device further includes coiling the tapered first wire. In one embodiment, coiling the tapered first wire includes winding the tapered first wire into a primary shape defined by a generally linear longitudinally extending coil. The coil in its primary shape is then coiled into a secondary shape defined by a spiral shaped coil having a series of loops axially spaced apart, forming a variable strength occluding device in accordance with one embodiment of the present invention.

In another embodiment, coiling the tapered first wire includes winding the tapered first wire into a primary shape defined by a spiral shaped primary coil having a plurality of axially spaced loops. In this embodiment, an elongate second wire having a first end and a second end is coiled into a secondary coil having a primary shape defined by a linear longitudinally extending coil. The secondary coil in its primary shape includes a second central axis extending between first and second ends of the secondary coil. In this embodiment, the secondary coil in its primary shape receives the first wire. For example, the longitudinally extending secondary coil may slide over the coiled first wire when the coiled first wire is in its primary shape, the secondary coil sliding over the loops defined by the primary shape of the coiled first wire and conforming to the primary shape of the coiled first wire (i.e., primary coil).

Alternatively, in the case of a nitinol tapered first wire, for example, the coiled first wire may be straightened before the secondary coil slides over the first wire. Due to its super-elastic or shape-memory properties, once within the secondary coil, the straightened first wire returns to its coiled configuration (i.e., primary shape) causing the secondary coil to conform to the primary shape of the coiled first wire (i.e., primary coil).

Thus, the secondary coil conforms to the spiral shaped primary coil having a plurality of axially spaced loops thereby defining a secondary shape of the secondary coil. The first and second axes coincide and the first and second ends of the first wire are adjacent the first and second ends of the secondary coil, respectively, when the secondary coil receives the first wire and forms its secondary shape.

The method further includes deploying the variable stiffness occluding device (404) at a desired point of occlusion in the body vessel. Deploying the variable stiffness occluding device includes introducing a guide catheter in the body vessel, passing an inner catheter through the guide catheter to position the inner catheter at the desired point of occlusion in the body vessel. The inner catheter includes a hub and the occluding device is loaded at the hub of the inner catheter. The occluding device is then advanced to a distal end of the inner catheter and deployed in the body vessel.

As a person skilled in the art will readily appreciate, the above description is meant as an illustration of the implementation of the principles of this invention. This description is not intended to limit the scope or application of this invention in that the invention is susceptible to modification variation and change, without departing from the spirit of this invention, as defined in the following claims.

1. An embolization coil having variable stiffness for occlusion of fluid flow through a lumen of a body vessel, the embolization coil comprising:
an elongate first wire having a proximal end and a distal end and a first central axis extending between the proximal and distal ends, the distal end having a first diameter and the proximal end having a second diameter smaller than the first diameter, the first wire formed with a tapered diameter defined by a continuously decreasing diameter along the first central axis from the distal end to the proximal end, wherein the first wire is coiled into a primary shape, wherein the tapered diameter of the coiled first wire provides the embolization coil with a continuously decreasing stiffness from the distal end to the proximal end.

2. The embolization coil of claim 1 wherein the primary shape of the coiled first wire is defined by a linear longitudinally extending coil, wherein the coiled first wire in the primary shape is helically wound into a secondary shape defined by a spiral shaped coil having a plurality of axially spaced loops.

3. The embolization coil of claim 1 wherein the tapered diameter of the first wire is formed by at least one of grinding and electrolytically tapering the wire.

4. The embolization coil of claim 1 further comprising fibers attached to the first wire and extending therefrom.

5. The embolization coil of claim 1 further comprising an elongate second wire having a proximal end and a distal end, wherein the second wire is wound into a second coil configured to receive the first wire.

6. The embolization coil of claim 5, wherein the primary shape of the coiled first wire is defined by a spiral shaped first coil having a plurality of axially spaced loops, wherein the second coil includes a primary shape defined by a linear longitudinally extending coil, wherein the second coil in its primary shape receives the first wire and conforms to the primary shape of the coiled first wire thereby forming a secondary shape of the second coil, wherein the secondary shape of the second coil is defined by a spiral shaped second coil having a plurality of axially spaced loops.

7. The embolization coil of claim 6 wherein the second coil includes a second central axis extending between proximal and distal ends of the second coil in its primary shape, wherein the first central axis of the first wire and the second central axis of the second coil coincide and wherein the proximal and distal ends of the first wire are adjacent the proximal and distal ends of the second coil, respectively, when the second coil receives the first wire and forms its secondary shape.

8. The embolization coil of claim 5 wherein the second wire is formed with a tapered diameter from the distal end to the proximal end.

9. The embolization coil of claim 5 further comprising fibers attached to the second wire and extending therefrom.

10. An embolization coil having variable stiffness for occlusion of fluid flow through a lumen of a body vessel, the embolization coil comprising:
an elongate first wire having a first end and a second end and a first central axis extending between the first and second ends, wherein the first wire tapers along the first central axis along its entire length from a larger diameter at the first end to a smaller diameter at the second end, wherein the tapered first wire is coiled into a primary shape, wherein the tapered first wire provides the embolization coil with a continuously decreasing stiffness from the first end to the second end.

11. The embolization coil of claim 10 wherein the primary shape of the coiled first wire is defined by a linear longitudinally extending coil, wherein the coiled first wire in the primary shape is helically wound into a secondary shape defined by a spiral shaped coil having a plurality of axially spaced loops.

12. The embolization coil of claim 10 further comprising fibers attached to the first wire and extending therefrom.

13. The embolization coil of claim 10 further comprising an elongate second wire having a first end and a second end, wherein the second wire is wound into a second coil configured to receive the first wire.

14. The embolization coil of claim 13 wherein the primary shape of the coiled first wire is defined by a spiral shaped first coil having a plurality of axially spaced loops, wherein the second coil includes a primary shape defined by a linear longitudinally extending coil, the second coil having a second central axis extending between first and second ends of the second coil in its primary shape, wherein the second coil in its primary shape receives the first wire and conforms to the primary shape of the coiled first wire thereby forming a secondary shape of the second coil, wherein the first and second axes coincide and wherein the first and second ends of the first wire are adjacent the first and second ends of the second coil, respectively, when the second coil receives the first wire and forms its secondary shape.

15. The embolization coil of claim 13 further comprising fibers attached to the second wire and extending therefrom.

16. The embolization coil of claim 10 wherein the first and second wires include at least one of nickel-based superalloys, stainless steel, platinum, iron, iridium, palladium, tungsten, gold, rhodium, rhenium, titanium, copper, cobalt, nitinol, and alloys thereof.

17. A method of occluding fluid flow through a lumen of a body vessel, the method comprising:
forming a variable stiffness embolization coil including:
tapering an elongate first wire having a first end and a second end and a first central axis extending between the first and second ends such that the first wire includes a continuously decreasing diameter along the first central axis from the first end to the second end;
coiling the tapered first wire, wherein the tapered first wire provides the embolization coil with a continuously decreasing stiffness from the first end to the second end; and
deploying the embolization coil at a desired point of occlusion in the body vessel.

18. The method of claim 17 wherein coiling the tapered first wire includes winding the tapered first wire into a primary shape defined by a linear longitudinally extending coil and winding the coiled first wire in its primary shape into a secondary shape defined by a spiral shaped coil having a plurality of axially spaced loops.

19. The method of claim 17 wherein coiling the tapered first wire includes winding the tapered first wire into a primary shape defined by a spiral shaped coil having a plurality of axially spaced loops, wherein forming the variable stiffness embolization coil further includes:
coiling an elongate second wire having a first end and a second end into a second coil having a primary shape defined by a linear longitudinally extending coil, the second coil in its primary shape having a second central axis extending between first and second ends of the second coil; and
receiving the first wire within the longitudinally extending second coil, wherein the second coil conforms to the primary shape of the coiled first wire thereby defining a secondary shape of the second coil, wherein the first and second axes coincide and wherein the first and second ends of the first wire are adjacent the first and second ends of the second coil, respectively, when the second coil receives the first wire and forms its secondary shape.

20. The method of claim 17 wherein tapering the first wire includes at least one of grinding and electrolytically tapering the first wire to form a continuously decreasing diameter along the first central axis.
